(19) United States
(12) Patent Application Publication
(10) Pub. No.: US 2024/0070875 A1
(43) Pub. Date: Feb. 29, 2024

(54) SYSTEMS AND METHODS FOR TRACKING OBJECTS CROSSING BODY WALLFOR OPERATIONS ASSOCIATED WITH A COMPUTER-ASSISTED SYSTEM

(71) Applicant: INTUITIVE SURGICAL OPERATIONS, INC., Sunnyvale, CA (US)

(72) Inventors: Dinesh Rabindran, San Jose, CA (US); Omid Mohareri, San Francisco, CA (US); Melanie Diane Yuan, Mountain View, CA (US); Kerry Wang, Saratoga, CA (US)

(21) Appl. No.: 18/259,833

(22) PCT Filed: Dec. 29, 2021
(86) PCT No.: PCT/US2021/065444

§ 371 Date: Jun. 29, 2023

Related U.S. Application Data
(60) Provisional application No. 63/132,421, filed on Dec. 30, 2020.

(51) Int. Cl. G06T 7/20; G06T 7/30; G06T 7/60

(52) U.S. Cl. CPC G06T 7/20; G06T 7/30; G06T 7/60; G06T 2207/30241

(57) ABSTRACT

A system includes a memory and a processing unit coupled to the memory. The processing unit is configured to receive first image data from a first image sensor exterior to a body, the first image data including data of an object; receive second image data from a second image sensor interior to the body, the second image data including data of the object; track the object moving across a body wall of the body based on the first and second image data to generate a tracking result to indicate a status of movement of the object across the body wall.

### CROSS-REFERENCE TO RELATED APPLICATIONS

This application claims the benefit of U.S. Provisional Application 63/132,421 filed Dec. 30, 2020, which is incorporated by reference herein in its entirety.

### FIELD

The present disclosure is directed to systems and methods for performing a robotic procedure, and more particularly to systems and methods for tracking objects moving across a body wall.

### BACKGROUND

More and more devices are being replaced with computer-assisted electronic devices. This is especially true in industrial, entertainment, educational, and other settings. As a medical example, the hospitals of today have large arrays of electronic devices being found in in patient's homes, examination rooms, operating rooms, interventional suites, intensive care wards, emergency rooms, and/or the like. Many of these electronic devices may be moved manually by nearby personnel, capable of semi-autonomous motion, and/or capable of autonomous motion. Some of these electronic devices also allow personnel to control the motion and/or operation of electronic devices using one or more input devices located at a user control system. As a specific example, minimally invasive, robotic telemedical systems permit medical personnel to perform operations on patients from bedside or remote locations.

When an electronic device is used to perform a task at a worksite, one or more image sensors (e.g., an optical camera, an ultrasound probe, an MRI sensor, a CT sensor, a fluoroscopic sensor) can capture images of the worksite that provide visual feedback to an operator who is monitoring and/or performing the task. The image sensor(s) may also be controllable to update a view of the worksite that is provided, via a display unit, to the operator. For example, the image sensor(s) or other tools could be attached to a repositionable structure that includes two or more links coupled together by one or more joints, where the repositionable structure can be moved (including through internal reconfiguration) to update a position and/or orientation of the image sensor or other tool at the worksite. In such a case, movement of the image sensor(s) or other tool may be controlled by the operator or another person or automatically.

As a specific medical example, a computer-assisted surgical system can be used to perform minimally invasive and/or other types of surgical procedures within an internal space of a body of a patient. For example, multiple medical tools may be coupled to manipulator arms of a computer-assisted surgical system, may be inserted into the patient by way of one or more ports (e.g., at natural orifices or incision sites) within a body wall of the patient, and may be robotically and/or teleoperatively controlled to perform a surgical procedure within the patient. Minimally invasive medical tools include tools such as therapeutic tools, diagnostic tools, and surgical tools. Minimally invasive medical tools may also include imaging tools with image sensors, such as endoscopic tools that provide a user with a field of view within the patient anatomy.

In various medical and non-medical procedures involving movement of objects across a body wall, tracking of the objects crossing body wall of the patient can allow operators to better target operations within the body, better determine whether the procedures have been performed successfully, and increase the efficiency or effectiveness of the procedure. Therefore, it is desirable to provide improved tracking of objects crossing the body wall, including in medical examples to improve tracking objects crossing the body walls of patients.

### SUMMARY

Embodiments of the invention are described by the claims that follow the description.

Consistent with some embodiments, an object tracking system includes a memory and a processing unit including one or more processors coupled to the memory. The processing unit is configured to receive first image data from a first image sensor exterior to a body, the first image data including data of an object; receive second image data from a second image sensor interior to the body, the second image data including data of the object; determine a first registration between the first image sensor and the second image sensor; track the object moving across a body wall of the body based on the first image data, the second image data, and the first registration; and generate a tracking result to indicate a status of movement of the object across the body wall.

Consistent with other embodiments, an object tracking method includes receiving first image data from a first image sensor exterior to a body, the first image data including data of an object; receiving second image data from a second image sensor interior to the body, the second image data including data of the object; determining a first registration between the first image sensor and the second image sensor; and generating a tracking result by tracking the object moving across a body wall of the body based on the first image data, the second image data, and the first registration, the tracking result indicating a status of movement of the object across the body wall.

Consistent with other embodiments, a non-transitory machine-readable medium comprising a plurality of machine-readable instructions which, when executed by one or more processors of a tracking system, are adapted to cause the one or more processors to perform a method. The method includes receiving first image data from a first image sensor exterior to a body, the first image data including data of an object; receiving second image data from a second image sensor interior to the body, the second image data including data of the object; determining a first registration between the first image sensor and the second image sensor; and generating a tracking result by tracking the object moving across a body wall of the body based on the first image data, the second image data, and the first registration, the tracking result indicating a status of movement of the object across the body wall.

Other embodiments include corresponding computer systems, apparatus, and computer programs recorded on one or more computer storage devices, each configured to perform the actions of the methods.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1A is a schematic view of a robotic system, in accordance with an embodiment of the present disclosure.

FIG. 1B is a perspective view of an operator's control console for a robotic system, in accordance with an embodiment of the present disclosure.

FIG. 2 is a perspective view of an operator's input controller, in accordance with an embodiment of the present disclosure.

FIG. 3 is a perspective view of a manipulator assembly with an image sensor system, in accordance with an embodiment of the present disclosure.

FIG. 4 illustrates a flowchart providing a method for tracking objects crossing a body wall of a body for operations associated with a computer-assisted system, in accordance with an embodiment of the present disclosure.

FIG. 5 illustrates a flowchart providing a method for tracking objects crossing a body wall of a body based on a tracking configuration, in accordance with an embodiment of the present disclosure.

FIG. 6 illustrates a flowchart providing a method for tracking a tool based on a tool tracking configuration, in accordance with an embodiment of the present disclosure.

FIG. 7 illustrates a flowchart providing a method for tracking a body portion based on a body portion tracking configuration, in accordance with an embodiment of the present disclosure.

FIG. 8 illustrates a flowchart providing a method for tracking an implant based on an implant tracking configuration, in accordance with an embodiment of the present disclosure.

FIGS. 9A, 9B, and 9C illustrates an environment including an exterior sensor and an interior sensor and the captured images respectively, in accordance with an embodiment of the present disclosure.

FIG. 10 illustrates an example display including a tracking result, in accordance with an embodiment of the present disclosure.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. In the following detailed description of the aspects of the disclosure, numerous specific details are set forth in order to provide a thorough understanding of the disclosed embodiments. However, it will be obvious to one skilled in the art that the embodiments of this disclosure may be practiced without these specific details. In other instances, well-known methods, procedures, components, and circuits have not been described in detail so as not to unnecessarily obscure aspects of the embodiments of the disclosure.

Any alterations and further modifications to the described devices, tools, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances, the same reference numbers are used throughout the drawings to refer to the same or like parts.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an embodiment using a surgical system. Knowledgeable persons will understand, however, that inventive aspects disclosed herein may be embodied and implemented in various ways, including robotic and, if applicable, non-robotic embodiments and embodiments. Robotic surgical embodiments are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (without return to a human or animal anatomy) and for procedures on human or animal cadavers. Further, these techniques can also be used for medical treatment or diagnosis procedures that include, or do not include, surgical aspects.

The embodiments below will describe various tools and portions of tools in terms of their state in three-dimensional space. As used herein, the term “position” refers to the location of an object or a portion of an object in a three-dimensional space (e.g., three degrees of translational freedom that can be described using changes in any appropriate coordinate system, such as in a Cartesian X, Y, Z coordinate system). As used herein, the term “orientation” refers to the rotational placement of an object or a portion of an object (three degrees of rotational freedom—e.g., which can be described using roll, pitch, and yaw). As used herein, the term “pose” refers to the position of an object or a portion of an object in at least one degree of translational freedom, and to the orientation of that object or that portion of that object in at least one degree of rotational freedom. For an asymmetric, rigid body in a three-dimensional space, a full pose can be described with six parameters in six total degrees of freedom.

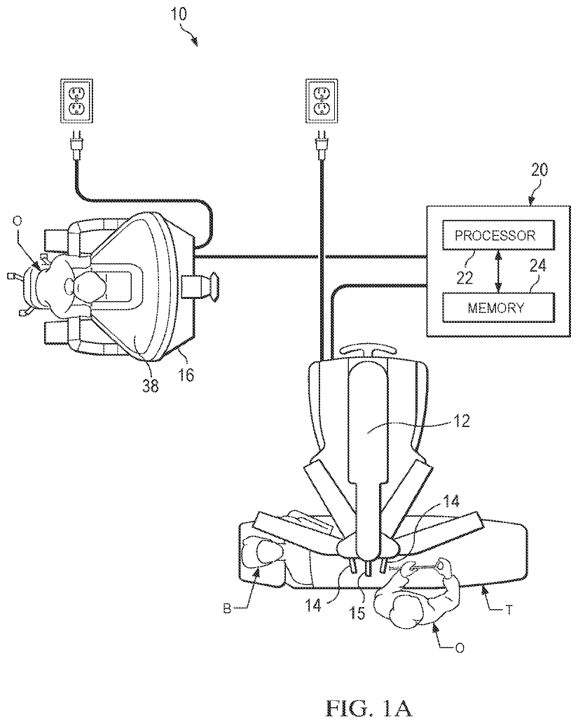

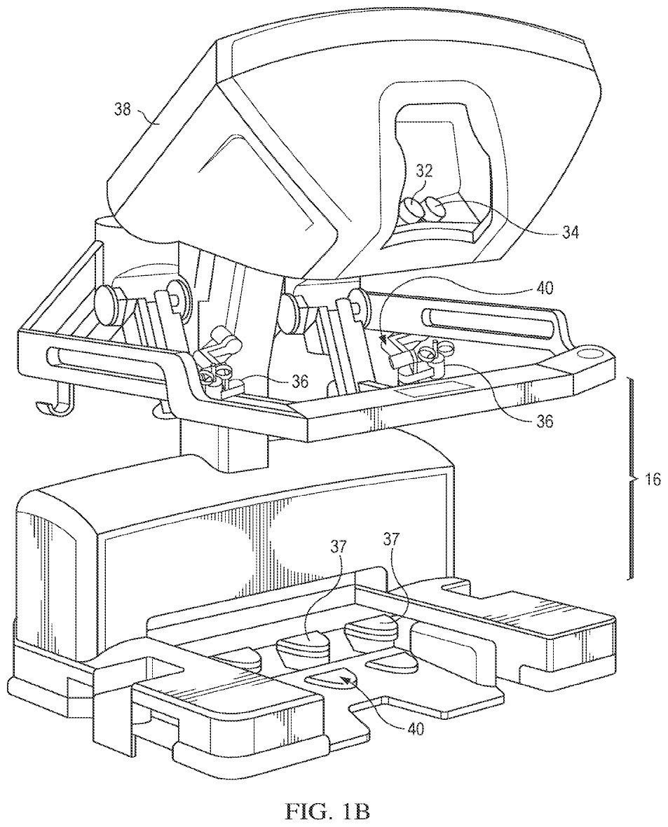

Referring to FIG. 1A of the drawings, an example robotic system is shown. Specifically, in FIG. 1A, a computer-aided, robotic system 10 that may be teleoperated. System 10 may be used in, for example, medical procedures, including diagnostic or therapeutic procedures. In some embodiments, manipulators or other parts of a robotic system may be controlled directly through manual interaction with the manipulators (or the other parts) themselves. “Teleoperated manipulators” as used in this application include manipulators that can be controlled only through teleoperation, and manipulators that can be controlled partially through teleoperation (e.g. direct manual control may be possible for parts of manipulators, or at different times or in different modes from teleoperation). Further, in some embodiments, a robotic system may be under the partial control of a computer programmed to perform the procedure or sub-procedure. In still other alternative embodiments, a fully automated robotic system, under the full control of a computer programmed to perform the procedure or sub-procedure, may be used to perform procedures or sub-procedures.

As shown in FIG. 1A, the robotic system 10 generally includes a manipulator assembly 12 mounted to or near table T on which a body B to which the manipulator assembly 12 is to perform a procedure is positioned (FIG. 1A shows the body B as a patient, for a medical example). The manipulator assemblies described herein often include one or more robotic manipulators and tools mounted thereon, although the term “manipulator assembly” also encompasses the manipulator without the tool mounted thereon. A tool 14 and a tool 15 are shown operably coupled to the manipulator assembly 12. For convenience within this disclosure, the tool 15 includes an image sensor, and may also be referred to as the imaging tool 15 when it does include an imaging sensor. The imaging tool 15 may comprise an endoscope. The imaging sensor of the imaging tool 15 may be based on optical imaging technology, ultrasonic imaging technology, or other technology (e.g. fluoroscopic, etc.). An operator input system 16 allows an operator O to view images of or representing the procedure site and to control the operation of the tool 14 and/or the tool 15.

The operator input system 16 for the robotic system 10 may be “mechanically grounded” by being connected to a base with linkages such as to an operator's console, or it may be “mechanically ungrounded” and not be thus connected. In the example shown in FIG. 1A, the operator input system 16 is connected to an operator's console 38 that is usually located in the same room as table T during the procedure. It should be understood, however, that the operator O can be located in a different room or a completely different building from the body B. The operator input system 16 generally includes one or more control device(s) for controlling the tool 14. The one or more control devices are also referred to herein as “input devices.”

The manipulator assembly 12 supports and manipulates the tool 14 while the operator O views the procedure site through the operator's console. An image of the procedure site can be obtained by the tool 15, such as in a medical example via an image sensor system comprising an endoscope. The number of tools 14 used at one time may vary with the procedure, the operator, the space constraints, and factors. The manipulator assembly 12 may include a kinematic structure of one or more non-servo controlled links (e.g., one or more links that may be manually positioned and locked in place) and a robotic manipulator. The manipulator assembly 12 includes a plurality of actuators that drive the tools 14. These actuators move in response to commands from the control system (e.g., control system 20). The actuators include drive systems which when coupled to the tools 14 may advance or retract the tools 14 through a body wall, move the distal end of the tools 14 in multiple degrees of freedom, or operate other functions of the tools 14 (e.g. applying energy, stapling, etc.). Movement of the tools 14 may include one, two, three or more degrees of translational freedom; one, two, three, or more degrees of rotational freedom; or other degrees of freedom (e.g. opening or closing jaws, movement of intermediate portions of the tools 14, etc.). In a medical example, the tools 14 may include end effectors each having a single working member such as a scalpel, a blunt blade, a needle, a suction irrigator, a endoscopic tip, an optical fiber, an electrode, and an electrocautery hook, or end effectors each having multiple working members, such as forceps, graspers, clip appliers, staplers, vessel sealers, electrocautery scissors, etc.

The robotic system 10 also includes a control system 20. The control system 20 includes at least one memory 24 and at least one processor 22, and typically a plurality of processors, for effecting control between the tool 14, the operator input system 16, and other auxiliary systems 26 which may include, for example, image sensor systems, audio systems, fluid delivery systems, display systems, illumination systems, steering control systems, irrigation systems, and/or suction systems. The one or more processors 22 of the control system 20 may be located in one location or located in different locations. In an example, the control system 20 may include a processor located in a manipulator assembly for processing image data from the image sensor systems. This option can also be covered, either here or wherever control system 20 is introduced. The control system 20 also includes programmed instructions (e.g., a computer-readable medium storing the instructions) to implement some or all of the methods described in accordance with aspects disclosed herein. While control system 20 is shown as a single block in the simplified schematic of FIG. 1A, the system may include two or more data processing circuits with one portion of the processing optionally being performed on or adjacent the manipulator assembly 12, another portion of the processing being performed at the operator input system 16, and the like. Any of a wide variety of centralized or distributed data processing architectures may be employed. Similarly, the programmed instructions may be implemented as a number of separate programs or subroutines, or they may be integrated into a number of other aspects of the teleoperational systems described herein. In one embodiment, control system 20 supports wireless communication protocols such as Bluetooth, IrDA, HomeRF, IEEE 802.11, DECT, and Wireless Telemetry.

In some embodiments, the control system 20 may include one or more servo controllers that receive force and/or torque feedback from the tool 14 or from the manipulator assembly 12. Responsive to the feedback, the servo controllers transmit signals to the operator input system 16. The servo controller(s) may also transmit signals that instruct the manipulator assembly 12 to move the tool(s) 14 and/or 15 which extends into an internal procedure site within the body via opening(s) in the body wall of the body. Any suitable conventional or specialized controller may be used. A controller may be separate from, or integrated with, manipulator assembly 12. In some medical embodiments, the controller and manipulator assembly are parts of an integrated system such as a teleoperational arm cart positioned proximate to a patient's body during a medical procedure.

The control system 20 can be coupled to the tool 15 and can include a processor to process captured images for subsequent display, such as to an operator O using the operator's console or wearing a head-mounted display system, on one or more stationary or movable monitors near the control system, or on another suitable display located locally and/or remotely. For example, where a stereoscopic or depth-capable image sensor is used, the control system 20 can process the captured images to present the operator with coordinated stereo images of the procedure site. Such coordination can include alignment between the stereo images and can include adjusting the stereo working distance of the stereoscopic endoscope.

In alternative embodiments, the robotic system may include more than one manipulator assembly and/or more than one operator input system. The exact number of manipulator assemblies will depend on the surgical procedure and the space constraints within the operating room, among other factors. The operator input systems may be collocated, or they may be positioned in separate locations. Multiple operator input systems allow more than one operator to control one or more manipulator assemblies in various combinations.

FIG. 1B is a perspective view of the operator's console 38. The operator's console 38 includes a left eye display 32 and a right eye display 34 for presenting the operator O with a coordinated stereo view of the operating environment. An operator input system 16 of the operator's console 38 includes one or more control devices 36, which in turn causes the manipulator assembly 12 to manipulate one or more tools 14 and/or 15. In a medical example, the control devices 36 may be used to operate tools 14 and/or 15 to, for example, move along translational or rotational degrees of freedom, close jawed end effectors, apply an electrical potential to an electrode, staple tissue, cut tissue, bend a joint along a shaft of a tool 14, 15, apply or suction fluid, or the like. In various alternatives, the control devices 36 may additionally or alternatively include one or more of a variety of input apparatuses, such as joystick devices, trackballs, data gloves, trigger-guns, voice recognition devices, touch screens, foot pedals, body motion sensors, presence sensors, and/or the like. In various embodiments, the control device(s) will be provided with more, fewer, or the same degrees of freedom as the tools commanded by the control device(s). Position, force, and tactile feedback sensors may be employed to transmit position, force, and tactile sensations associated with the tools 14, 15 back to the operator O through the control devices 36.

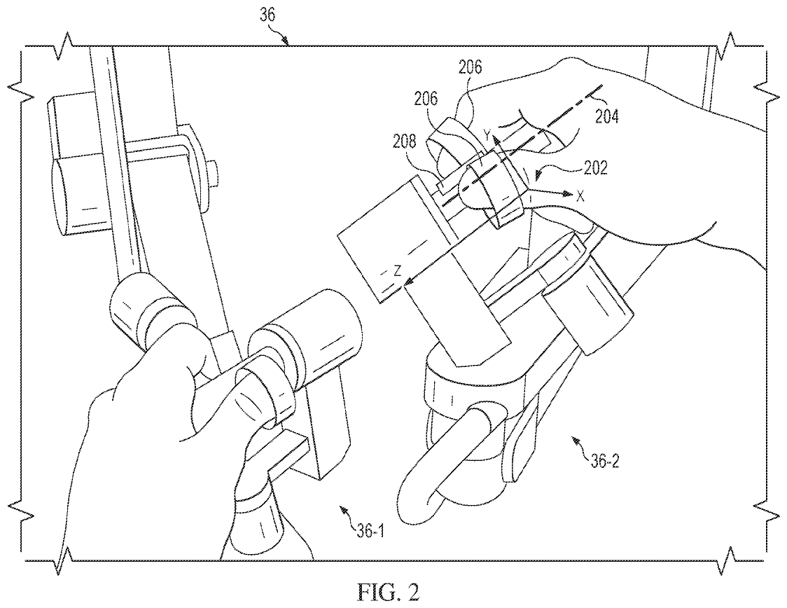

As shown in FIG. 2, in some embodiments, control devices 36 may include none, one, or more grip inputs 206 and/or switches 208. As illustrated in the example of FIG. 2, a master reference frame 202 associated with the control device 36, denoted as ml, is provided. The Z axis of the master reference frame 202 is parallel to an axis of symmetry 204 of the control device 36. The X and Y axes of the master reference frame 202 extend perpendicularly from the axis of symmetry 204.

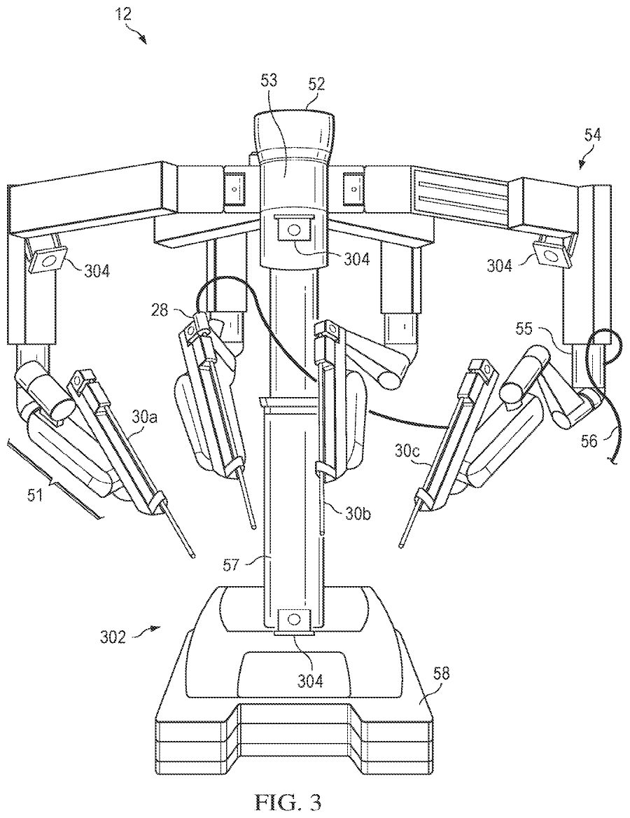

Referring to FIG. 3, illustrated is a perspective view of one embodiment of a manipulator assembly 12 (e.g., configured in the form of a cart that is located near the body B during a procedure). The manipulator assembly 12 shown provides for the manipulation of three tools 30a, 30b, 30c (e.g., similar to tools 14) and another tool 28 including an image sensor (e.g., similar to tool 15) used for the capture of images of the workpiece or of the site of the procedure (also called “work site”). The tool 28 may transmit signals over a cable 56 to the control system 20. Manipulation of the tools 30a, 30b, 30c, 28 is provided by robotic manipulators having a number of joints. The tool 28 and the tools 30a-c can be positioned and manipulated through openings in the body. The robotic manipulators and the tools 28, 30a-c can be manipulated such that a kinematic remote center is maintained, each robotic manipulator or tool is pivoted about its associated remote center during operations. In some embodiment, the kinematic remote center is maintained at an opening. In alternative embodiments, the kinematic remote center is maintained at a point other than the opening. For example, when an external access port is used to facilitate entry of the tool into the body, the remote center of motion may be outside the body at the entry into the access port, or somewhere in between the access port entry and an incision in the body. Images of the work site can include images of the tools 30a-c when they are positioned within the field-of-view of the image sensor of the tool 28.

The manipulator assembly 12 includes a movable, lockable, and drivable base 58. The base 58 is connected to a telescoping column 57, which allows for adjustment of the height of the manipulator arms 54. The manipulator arms 54 may include a rotating joint 55 that both rotates and translates parallel to the column 57. The manipulator arms 54 may be connected to a rotatable platform 53. The manipulator assembly 12 may also include a telescoping horizontal cantilever 52 for moving the platform 53 in a horizontal direction.

In the present example, each of the manipulator arms 54 includes a manipulator 51. The manipulator 51 may connect directly to a medical tool 14 and may or may not be teleoperatable.

Endoscopic and other image sensors (e.g., that of tool 15) may be provided in a variety of configurations, including ones having structures that are rigid, bendable or extendable at certain sections, or flexible. Optical image sensors may include a relay lens or optical fiber system for transmitting an image from a distal end to a proximal end of the tool comprising the image sensor. Digital-image based optical image sensors may use a distal digital sensor such as a one or more charge-coupled device (CCD) or a complementary metal oxide semiconductor (CMOS) device. Image sensors may also utilize other imaging techniques such as ultrasonic, infrared, hyperspectral, and fluoroscopic technologies. Image sensors may provide two- or three-dimensional images. Two-dimensional images may provide limited depth perception.

In various embodiments, the manipulator assembly 12 may be configured in the form of a cart, or be mounted to a table, a ceiling, a wall, a floor, etc. In various embodiments, a manipulator assembly 12 may comprise a single manipulator arm, or multiple manipulator arms as shown in FIG. 3.

In the example of FIG. 3, one or more image sensors 304 of an image sensor system 302 (also referred to as exterior sensor system 302, or sensor system 302) are attached to the manipulator assembly 12. In various examples, one or more sensors of the image sensor system 302 may be located at various locations (e.g., the cart, the links, the joints, etc.) of the manipulator assembly 12. In some embodiments, one or more image sensors of the image senor system 302 may be attached to the manipulator assembly 12, or be integrated into the manipulator assembly 12.

In various embodiments, the exterior sensor system 302 may include image sensors attached to manipulator assembly, imaging sensors attached to a structure not part of the manipulator assembly, and/or a combination thereof. Those image sensors may provide image data of the same scene (e.g., the patient on the table) from different points of view. The points of view of different image sensors may be chosen to maximize the spatial-temporal information of objects of interest in the scene. The points of view of the image sensors may be fixed or variable depending on the application. The exterior sensor system 302 may provide image data regarding environment exterior to the body on which an operation is to be performed (e.g., provide to control system 20). The exterior sensor system 302 may include one or more sensors including, for example, image sensors including optical sensors, depth sensors, time of flight sensors, any other suitable sensors, and/or a combination thereof. In some examples, the optical sensors include cameras that detect visible light or non-visible light. In some examples, the imaging sensors may have on-board motion sensors such as inertial measurement units (IMUs) or accelerometers, or have the capability to estimate motion of the cart in addition to the degrees of freedom on the manipulator assembly. In some examples, the system may use these additional motion sensors to perform visual simultaneous localization and mapping (vSLAM) to build a map of the scene as the cart moves into its final position near the operating table. The image sensors may capture images of environment exterior to the patient. An image processing unit (e.g., in control system 20) may process the resulting image data to identify, locate, and/or track particular objects (e.g., tools, removed body portions, implants, etc.). For example, the particular objects may be identified, located, and/or tracked by markings, colors, shapes, any other suitable features for identification, and/or a combination thereof. In some embodiments, the control system 20 may identify, locate, and/or track objects using an artificial intelligence (AI) system. In some examples, the AI may consist of a deep neural network trained to classify and segment said objects temporally and spatially. Depth information may be provided by integrated or separate depth sensors, triangulation through use of multiple cameras or stereoscopic cameras, or any appropriate technique. In some examples, time of flight sensors include laser rangefinder, LED rangefinder, lidar, radar, etc. In embodiments when the sensors include optical sensors or time of flight sensors, the control system may detect and process occlusion, because those sensors may provide information of an exterior object (an object exterior to the body) only when they are able to view at least a portion of the exterior object. The control system may also process the information (e.g., image data, depth data) to provide a three-dimensional model of the environment exterior to the patient, including the identified objects. In some embodiments, image data from several imaging sensors may be fused together before control system 20 processes it. In some examples, such data may be fused by registering one camera to the other using the knowledge of the kinematic chain between the cameras.

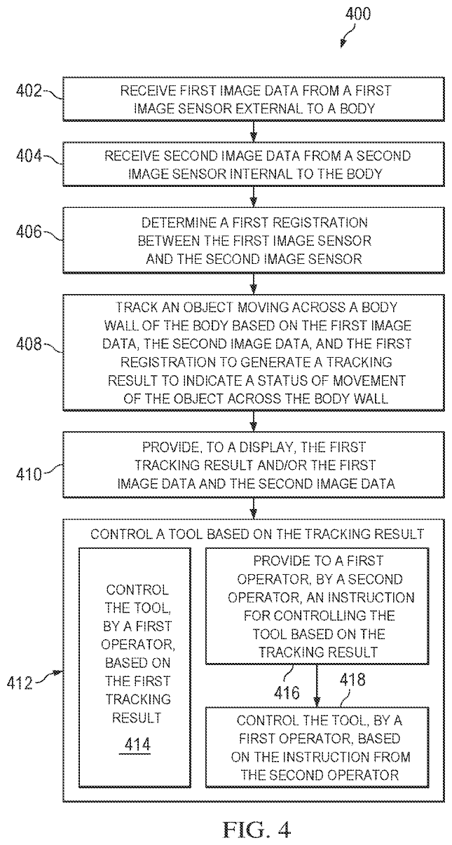

Referring to FIGS. 4-10, a control system (e.g. the control system 20 for the example of FIG. 1A) may perform tracking of an object (e.g., a tool, a body portion, an implant, etc.) using images exterior and interior to a body (e.g., of a patient in a medical example). The control system may receive one or more images from one or more image sensors 304 of the exterior image sensor system 302 that senses images exterior to the body, and one or more images from an image sensor of the tool 28 (e.g., similar to tool 15) that senses images interior to the body. The control system may perform a registration to determine a registration (e.g., including alignment relationships of partial or full degree of freedoms) between images provided by these image sensors, and transform these image data to a common reference frame. In some examples, such a registration may involve determining the 3 degrees of freedom translation and 3 degrees of freedom rotation transformation (or a subset of these degrees of freedom) between the image sensors' field(s) of view and the common reference frame. Using the transformed image data from image sensors exterior and interior to a body, the control system may track the object's movement across a body wall of the body, and generate a tracking result to indicate a status of movement of the object across the body wall. In various embodiments, the tracking result may indicate the object to track, whether the object was moved from the exterior to the interior to the body or from the interior to the exterior to the body, a moving direction of the object, a total number of sub-portions of the object that have been moved, a location of the object relative to the body wall, an amount or shape of the object that has moved or is moving across the body wall, an integrity metric of the object after it has moved across the body wall, whether that object has moved across the body wall, and the like.

In various embodiments, the control system may determine a tracking configuration based on the object to track and the operation anticipated or in process, and perform the tracking using the tracking configuration. For example, the control system may determine a tracking configuration based on a type of the object (e.g., a tool, a body portion, an implant, etc.). As another example, the operation tracked may include the object entering or exiting the body (e.g., a tool entering and/or exiting the body; removing and/or transplanting a body portion; removing, placing, and/or replacing an implant, etc.).

Referring to the example of FIG. 4, a flowchart provides a method 400 for tracking objects moving across a body wall of a body during an operation using image data from image sensors both exterior and interior to the body. The method 400 begins at process 402, where a control system receives sensor data including first image data from a first image sensor exterior to a body. The first image data includes an object to track. Such an object to track may be identified, in the first image data, by markings, colors, shapes, size/dimensions, any other suitable features, associations with equipment that may be interacting with the object, attributes or features identified by a machine learning model, and/or a combination thereof.

The method 400 may proceed to process 404, where the control system receives sensor data including second image data from a second image sensor (e.g., an image sensor system of medical tool 14 or 15) interior to the body. The second image data may include the object to track or a representation/model of the object to track. The object to track may be identified, in the second image data, by markings, colors, shapes, size/dimensions, any other suitable features, associations with equipment that may be interacting with the object, attributes or features identified by a machine learning model, and/or a combination thereof. In some embodiments, the control system may use the same feature(s) for identifying the object to track in the first and second image data. Alternatively, in some embodiments, the control system may use different feature(s) for identifying the object to track in the first and second image data, e.g., based on the different image properties (e.g., imaging conditions, image resolution, etc.) of the first and second image data. In some embodiments, the views for an exterior image sensor and an interior image sensor may be mutually exclusive (e.g., one sees inside the body while the other sees outside the body), and the tracked object may not be included in both first image data and second image data captured at the same time. In those embodiments, a synthetic (e.g., based on a model of the object to track) overlay of the object to track (e.g., based on information from one image data) may be provided in the other image data where the object to track is not directly visible.

The method 400 may proceed to process 406, where the control system determines a registration between the first and second image sensors. In some embodiments, the registration is performed by registering the image sensors to manipulators coupled to the image sensors, and registering the manipulators to each other. Various image registration methods may also be used by the control system to determine such an alignment relationship using the first and second image data. In some embodiments, the registration is performed further using additional image data (e.g., pre- and intra-operative image data, computed patient mesh, etc.), and the registered set of images includes first and second image data from the first and second image sensors and those registered additional image data. The control system may transform the first and/or second image data according to the alignment relationship to a common reference frame. In some embodiments, the common reference frame may be a 3D coordinate system coincident with the reference frame of either imaging sensor, the 3D coordinate system of the manipulator assembly, or 2D image planes of either image sensor.

The method 400 may proceed to process 408, where the control system tracks the object moving across a body wall of the body based on the first and second image data and the first registration to generate a first tracking result to indicate whether the object has moved across the body wall. In an example, the control system may generate one or more movement paths of the object, wherein first and second image data include multiple images or video images. In some embodiments, when the object to track is not present directly in either (the first or second) image data, or when the object to track is present but occluded in either the first or second image data, an estimate of its position and motion may be generated based on its past recorded positions and motions in either image data. In some examples, such estimation may be used to track objects as they transition in-vivo to ex-vivo (or vice-versa) through a lumen, cavity, or region such as a cannula that is not within the field of view of the first or second image sensor.

The method 400 may proceed to process 410, wherein the control system provides, to one or more displays, the tracking result and/or the first and second image data to a display. In some embodiments, the tracking result and/or the first and second image data are sent to multiple displays (e.g., a monitor, a smartphone, a tablet, a touch screen, etc.) for a same operator. In some embodiments, the tracking result and/or the first and second image data are sent to displays for multiple operators, such that each of those operators may have the same information regarding the tracking of the object. Such display of the tracking result and/or the first and second image data to different operators facilitates efficient collaboration between different operators. In some embodiments, the control system determines operation options and/or recommendations to the operators based on the tracking result, and provides the options and/or recommendations to the operators (e.g., using the one or more displays, sending a text message, voice prompts, graphical overlays, etc.). The tracking result or recommendations may be presented on a relevant display at a relevant time for a relevant operator based on the task being performed.

In some embodiments, the display may be used to provide an operation or task status indicating a status and progress of a particular operation or task that is determined based on the tracking result. Such an operation status may be presented to operators in various ways, e.g., using text, progress bar, auditory sounds or speech, video, etc. In an example, the tracking result is used to determine whether a tool has reached a target. Such a determination may be determined based on various information of the tracking result (e.g., whether the tool is inserted to a certain depth, is fully within body wall, is retracted to a certain amount, is fully removed from within the body wall, any other suitable information, and/or a combination thereof). In another example, the task status provides a dynamically updated count of the total number of tools and accessories that passes across the body wall. In yet another example, the task status includes a completion status of a task (e.g. removal of all sponges that was put inside the body wall for a sponge removal task). In yet another example, the task status indicates whether the object crossing the body wall is going in the correct direction (e.g. entering vs. exiting, etc.) relative to the body wall. In yet another example, the operation or task status indicates the time taken by the object to move from a source location of interest to a destination location of interest.

The method 400 may proceed to process 412, wherein a tool is controlled based on the tracking result. In various embodiments, the tool may be the same as the object being tracked (e.g., at process 408), or may be different from the object being tracked. In some embodiments, as shown in process 414, the tool is operated by a first operator (e.g., a surgeon or other clinician, or a patient side assistant) based on the first tracking result. The first operator may be provided the tracking result (alone or together with the first and second image data) using one or more displays, and may control the tool based on the tracking result. Alternatively, in some embodiments, as shown at process 416, a second operator (e.g., a surgeon or other clinician, or another assistant) may be provided the tracking result (alone or together with the first and second image data) using a display (e.g., different from the display for the first operator), and may provide instructions (e.g., moving the tool in a certain direction, performing a particular operation using the tool, coordinating the tool, etc.) to the first operator. At process 418, after receiving the instruction from the second operator, the first operator may control the tool based on the instruction from the second operator. In some embodiments, the registration step of processing 406 may enable the second operator and first operator to communicate about the tracking result, or to coordinate actions in response to the tracking result, in a common reference frame.

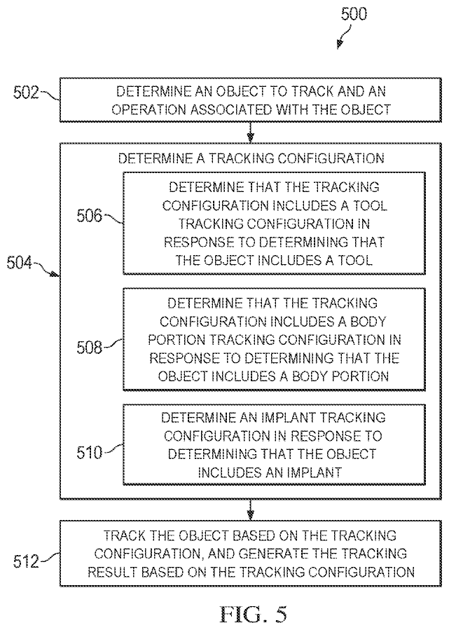

Referring to the example of FIG. 5, a method 500 for tracking an object (e.g., at process 408 of FIG. 4) across a body wall based on a tracking configuration is illustrated. The method 500 begins at process 502, where the control system determines the object to track and the associated operation. The object to track and the associated operation may be provided by an operator using an input device.

The method 500 may proceed to process 504, where the control system determines a tracking configuration. e.g., based on the object to track, the associated operation, and/or a combination thereof. For example, at process 506, the control system determines that the tracking configuration includes a tool tracking configuration in response to determining that the object to track includes a first tool for performing the operation. For further example, at process 508, the control system determines that the tracking configuration includes a body portion tracking configuration in response to determining that the object to track includes a body portion (e.g., a tissue sample, an organ to be removed, an organ to be placed for organ transplantation, etc.). Yet in another example, at process 510, the control system determines that the tracking configuration includes an implant tracking configuration in response to determining that the object to track includes an implant.

The method 500 may proceed to process 512, where the control system may perform the tracking of the object based on the tracking configuration, including e.g., performing tracking steps of the tracking configuration. Further, the control system may determine the tracking result based on the tracking configuration,

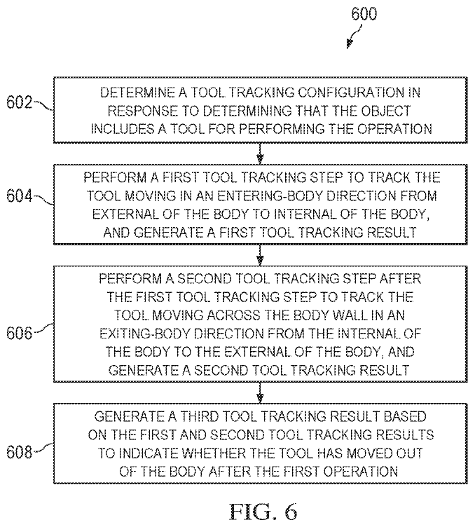

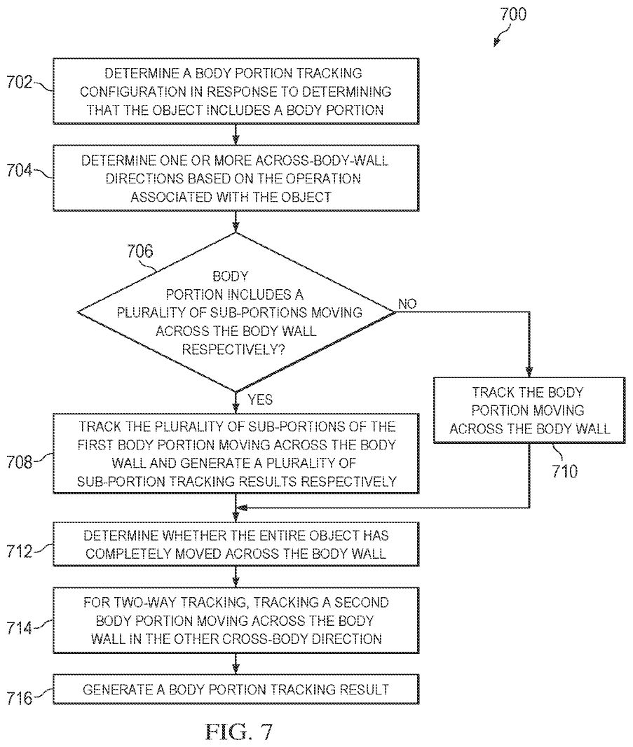

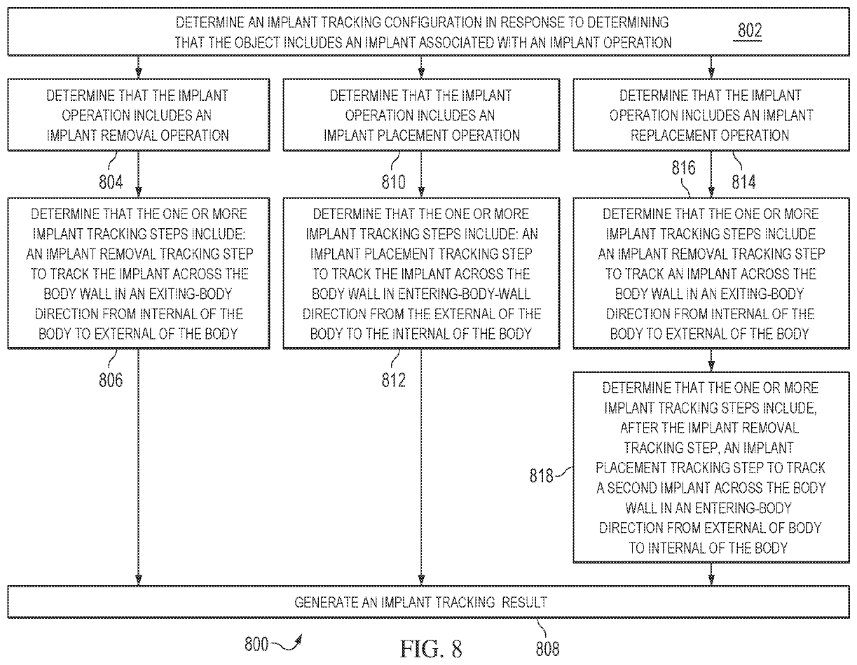

Referring to the examples of FIGS. 6, 7, and 8, illustrated are methods 600, 700, and 800 for performing a tracking based on specific example tracking configurations (e.g., at process 512). specifically, FIG. 6 and method 600 illustrate a tool tracking configuration, FIG. 7 and method 700 illustrate a body portion tracking configuration, and FIG. 8 and method 800 illustrate an implant tracking configuration. It is noted that these tracking configurations, objects to track, and associated operations are exemplary only, and other tracking configurations, objects to track, and associated operations may be used. For example, at process 512, an accessory tracking configuration may be determined based on determining that the object includes an accessory or a portion thereof (e.g., a sponge, a suture needle, a blade, a cautery tip, a dental rolls, an endo fog, a hypo needle, a lap sponge, a Penrose drain, a Raytec, a robotic scissor tip, a robotic stapler sheath, a ruler, a pediatric feeling tube, a Cottonoid, a smoke evacuator tip, etc.) for performing an associated operation. The accessory tracking configuration may be substantially similar to the tool tracking configuration described below with reference to FIG. 6.

Referring to the example of FIG. 6, the method 600 describes performing tracking based on a tool tracking configuration. The method 600 begins at process 602, where the control system determines a tool tracking configuration in response to determining that the object includes a first tool for performing the first operation. The tool tracking configuration includes a first tool tracking step to track the first tool moving in an entering-body direction from the exterior to the body to the interior to the body, and generate a first tool tracking result, and a second tool tracking step after the first tool tracking step to track the first tool moving across the body wall in an exiting-body direction from the interior to the body to the exterior to the body. In some examples, the operator may be interested in only one of those tool tracking steps, and the tool tracking configuration may be set accordingly.

The method 600 may proceed to process 604, where the control system performs a first tool tracking step to track the first tool moving in an entering-body direction from exterior to the body to interior to the body. The control system may generate a first tool tracking result indicating whether the first tool has moved across the body wall in the entering-body direction.

In some embodiments, the first tool tracking result includes a tool integrity confirmation indicating integrity of the tool after moving across the body wall. In an example, such a tool integrity is determined based on a measurement difference by comparing a measurement of the tool after it has moved across the body way with a reference measurement (e.g., a measurement determined before the operation or before moving across the body wall). In some embodiments, the measurement is determined based on a boundary of the tool and a computation of its linear dimensions (length, breadth, angular position, pose, etc.), area in a plane, or volume of a part or whole of the tool. The boundary may be provided by an operator, or may be determined by the control system automatically (e.g., using object boundary detection algorithms in image processing, machine learning, any other suitable algorithms, and/or a combination thereof) using the first image data, second image data, first alignment data, kinematics data, and/or additional image data. In some examples, when a plurality of tools is being tracked, the count or number of tools exiting and entering the body across the body maybe tracked.

The method 600 may proceed to process 606, where the control system performs a second tool tracking step after the first tool tracking step to track the first tool moving across the body wall in an exiting-body direction. The control system may generate a second tool tracking result indicating whether the first tool has moved across the body wall in the exiting-body direction. In some embodiments, the second tool tracking result includes a tool integrity confirmation indicating integrity of the tool after moving across the body wall, e.g., based on a measurement difference between the measurement of the first tool, after it moves across the body wall in an exiting-body direction, with a reference measurement.

The method 600 may proceed to process 608 to generate a third tool tracking result based on the first and second tool tracking results to indicate whether the tool has moved out of the body after the operation.

Referring to the example of FIG. 7, the method 700 describes performing tracking based on a body portion tracking configuration. The method 700 begins at process 702, where the control system determines a body portion tracking configuration in response to determining that the object includes a first body portion. The method 700 may proceed to process 704, where the control system determines one or more across-body-wall directions (e.g., entering-body-wall direction, exiting-body-wall direction, and/or a combination thereof) based on the operation associated with the body portion. For example, if the operation is a body portion removal operation (e.g., a body tissue extraction operation, an organ removal operation), then the one or more across-body-wall directions includes an exiting-body-wall direction. For further example, if the operation is a body portion placement operation (e.g., an organ placement operation to place an organ of another person in the patient), then the one or more across-body-wall directions includes an entering-body-wall direction. Yet in another example, if the operation is an organ implantation operation including an organ removal operation to remove a first organ from the patient, followed by an organ placement operation to place a second organ in the patient, then the one or more across-body-wall directions includes an exiting-body-wall direction followed by an entering-body-wall direction

The method 700 may proceed to process 706, where the control system determines whether the body portion includes a plurality of sub-portions to be moved across the body wall respectively. In response to a determination of having a plurality of sub-portions, the method 700 proceeds to process 708, where the control system tracks each of the plurality of sub-portions moving across the body wall and generates a plurality of sub-portion tracking results respectively. Alternatively, in response to a determination of not having a plurality of sub-portions, the method 700 proceeds to process 710, where the control system tracks the body portion moving across the body wall.

The method 700 may proceed to process 712, where the control system determines whether the entire object has completely moved across the body wall. In some embodiments, such an indication is determined based on a measurement difference between the object after it has moved across the body wall and a reference measurement. In the case where the body portion includes a plurality of sub-portions, the measurement may be determined based on individual sub-portion measurements (e.g., by combining all the sub-portion measurements), aggregation of those individual sub-portion measurements, and/or a combination thereof. In some examples, the measurement might include the object's linear dimensions (length, breadth, angular position, pose, etc.), area in a plane, or volume of a part or whole of the object, or shape of the object. In yet other examples, the measurement might include a count of number of sub-portions moving across the body wall. In some examples, the measurement might include an estimated weight, mechanical property, shade of color of the object, other suitable measurements, and/or a combination thereof. In some examples, the measurement might be a likelihood of the object belonging to a class of objects that an AI is trained to segment and classify. In some examples, the aggregation of individual sub-portion measurements might include a summation of individual measurements or a total count of sub-portions. In some examples, the aggregation of individual sub-portion measurements may include one of a variety of statistics such as determining a weighted mean or other measure of central tendency of individual sub-portion measurements. In some examples, a new measurement can be inferred from the sub-portion measurements; for example, an anatomical feature may be labeled or identified once the control system has processed the likelihood measurements from each sub-portion.

The method 700 may proceed to process 714, where the control system determines that tracking directions includes both entering- and existing-body-wall directions (e.g., for an organ transplantation operation). In that case, the control system may perform tracking of a second body portion moving across the body way in the other across-body-wall direction.

The method 700 may proceed to process 716, where the control system may generate a body portion tracking result, including, for example, an object confirmation indicating that the entire object has completed moved across the body wall. Such an object configuration may be determined by matching a total number of the sub-portions of the body portion in the body and outside of the body.

Referring to the example of FIG. 8, the method 800 describes performing tracking based on an implant tracking configuration. The method 800 begins at process 802, where the control system determines an implant tracking configuration in response to determining that the object includes an implant for an implant operation.

In some embodiments, the method 800 proceeds to process 804, where the control system determines that the implant operation includes an implant removal operation. The method 800 may proceed to process 806, where the control system determine that the one or more implant tracking steps include an implant removal tracking step to track the first implant across the body wall in an exiting-body direction from interior to the body to exterior to the body.

In some embodiments, the method 800 proceeds to process 810, where the control system determines that the implant operation includes an implant placement operation. The method 800 proceeds to process 812, where the control system determines that the tracking steps include an implant placement tracking step in the entering-body-wall direction.

In some embodiments, the method 800 proceeds to process 814, where the control system determines that the implant operation includes an implant replacement operation. The method 800 proceeds to process 816, determine that the tracking steps include an implant removal step followed by an implant placement at 818.

The method 800 may then proceed to process 808 and generate an implant tracking result indicating whether the implant has moved across-the-body-wall, in the determined one or more across-body-wall directions.

The method 800 then proceed to process 808, where the control system generates an implant tracking result.

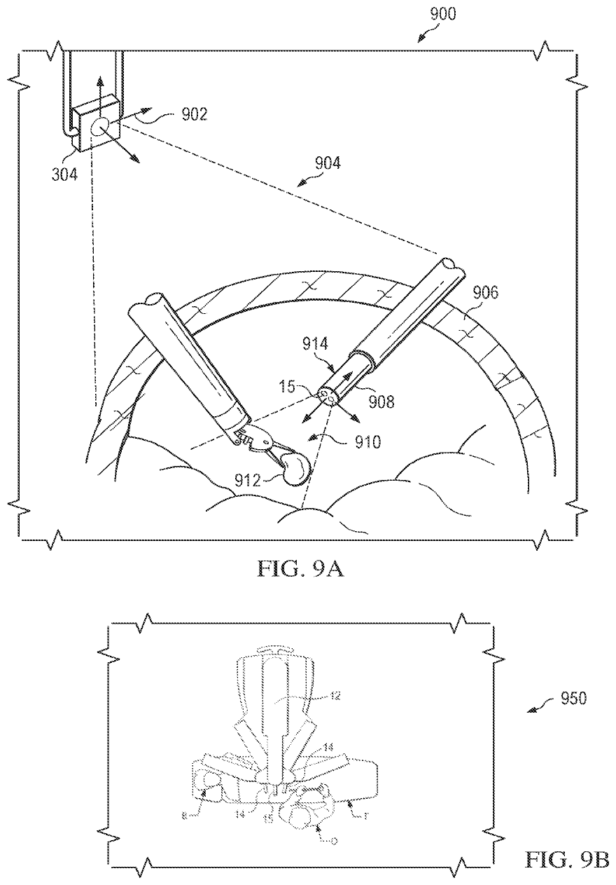

Referring to the examples of FIGS. 9A, 9B, and 9C, illustrated in FIG. 9A is an example environment 900 including an exterior image sensor and an interior image sensor. FIG. 9B illustrates an exterior image 950 including the body B from the exterior image sensor of FIG. 9A. FIG. 9C illustrates an interior image 960 from the interior image sensor of FIG. 9A. The exterior image 950 and the interior image 960 may be captured about at the same time or at different times (e.g., different times at a same operation, different times at different operations, etc.).

In the example of FIG. 9A, the environment 900 includes an exterior image sensor 304 (e.g., attached to/mounted to a manipulator assembly as in the example of FIG. 3, a ceiling, a wall, etc.), and an interior image sensor 914 (e.g., mounted on the tool 15) positioned to provide an interior image. The exterior image sensor 304 is associated with an exterior image sensor reference frame 902, and has a field of view 904. The exterior image sensor 304 may provide an exterior image (e.g., exterior image 950 of FIG. 9B) of the patient B, where a manipulator assembly 12 is used to perform surgery using tools including a tool 15, and the tool 15 (e.g., an endoscope) is visible in the exterior image. While the example exterior image 950 of FIG. 9B is from an exterior image sensor 304 mounted to a ceiling, the exterior image sensor 304 may be located at any appreciate location. In an embodiment where the exterior image sensor 304 is located on an arm of the manipulator assembly (e.g., as shown in the example of FIG. 3), the exterior image 950 provides an egocentric view from the manipulator assembly, and may provide an effective view for tracking the object 912.

As shown in FIG. 9A, interior image sensor 914 positioned to provide an interior image in the environment 900 is associated with an interior image sensor reference system 908 and has a field of view 910. The interior image sensor 914 may provide an interior image (e.g., interior image 960 of FIG. 9C) of the patient and the operation site inside the body wall 906 of the patient. An object 912 may be identified as the object to track. As shown in FIGS. 9A, 9B, and 9C, the object 912 is in the field of view 910 of the tool 15 at time T1, and is in the interior image 960 but not in the exterior image 950 at time T1. In an example, the object 912 moves through the body wall 906 at time T2. As such, at time T2, the object 912 is in the field of view 904 of the exterior image sensor 304, and is in the exterior image but not in the interior image at time T2.

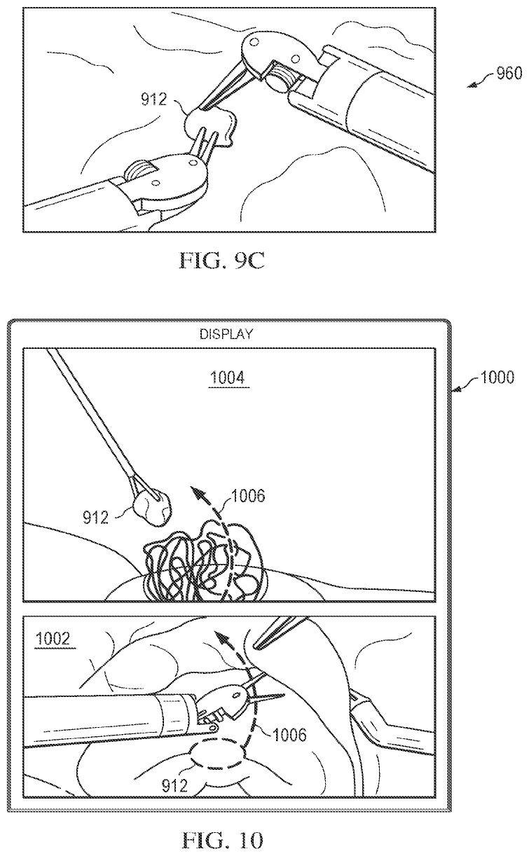

Referring to the example of FIG. 10, an example display 1000 displaying a tracking result of an object 912 is illustrated. The display 1000 includes an interior image 1002 (e.g., interior image 960 transformed from the exterior image sensor reference frame 902 to a common reference frame), including the object 912 with a timestamp T1. The display 1000 includes an exterior image 1004 (e.g., exterior image 950 transformed from the exterior image sensor reference frame 902 to a common reference frame), including the object 912 at a timestamp T2. A tracking path 1006 indicates the object 912's movement path across the body wall. In some examples, the display 1000 may include a message about the tracking result, e.g., indicating the number of sub-portions that have moved across the body wall out of the total number of sub-portions of the object (e.g., “2 out of a total of 6 sub-portions have been removed”). In some examples, the message may be accompanied by audio cues to inform the user.

In this disclosure, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various special device positions and orientations. The combination of a body's position and orientation define the body's pose.

Similarly, geometric terms, such as “parallel” and “perpendicular” are not intended to require absolute mathematical precision, unless the context indicates otherwise. Instead, such geometric terms allow for variations due to manufacturing or equivalent functions.

In addition, the singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. The auxiliary verb “may” likewise implies that a feature, step, operation, element, or component is optional.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, tools, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. An object tracking system comprising:
a memory;
a processing unit including one or more processors coupled to the memory, the processing unit configured to:
receive first image data from a first image sensor exterior to a body, the first image data including data of an object;
receive second image data from a second image sensor interior to the body, the second image data including data of the object;
determine a first registration between the first image sensor and the second image sensor;
track the object moving across a body wall of the body based on the first image data, the second image data, and the first registration; and
generate a tracking result to indicate a status of movement of the object across the body wall.

2. The system of claim 1, wherein:
the processing unit is further configured to determine a tracking configuration based on a type of the object, the tracking configuration including one or more tracking steps;
the processing unit is configured to track the object by performing the one or more tracking steps; and
the processing unit is configured to generate the tracking result based on the tracking configuration.

3. The system of claim 1, wherein:
the processing unit is further configured to determine a tool tracking configuration in response to determining that the object includes a tool for performing an operation in the body;
the tool tracking configuration includes a tool tracking step to track the tool moving in an entering-body direction or an exiting-body direction, the entering-body direction being from exterior to the body to interior to the body, and the exiting-body direction being from the interior to the body to the exterior to the body;
the processing unit is configured to track the object by performing at least the tool tracking step; and
the processing unit is configured to generate the tracking result based on the tool tracking configuration.

4. The system of claim 1, wherein:
the processing unit is further configured to determine a tool tracking configuration in response to determining that the object includes a tool for performing an operation in the body, the tool tracking configuration including a first tool tracking step and a second tool tracking step, the first tool tracking step for tracking the tool moving in an entering-body direction from exterior to the body to interior to the body, and the second tool tracking step for tracking the tool moving in an exiting-body direction from the interior to the body to the exterior to the body;
the processing unit is configured to track the object by performing at least the first and second tool tracking steps;
the processing unit is configured to generate the tracking result based on the tool tracking configuration;
the status indicates a motion status of the tool moving out of the body after the operation.

5. The system of claim 1, wherein:
the processing unit is further configured to determine a body portion tracking configuration in response to determining that the object includes a body portion;
the body portion tracking configuration includes a body portion tracking step to track the body portion;
the processing unit is configured to track the object by performing at least the body portion tracking step; and
the processing unit is configured to generate the tracking result based on the body portion tracking configuration.

6. The system of claim 5, wherein:
in response to the body portion including a plurality of sub-portions, the body portion tracking step comprises tracking the plurality of sub-portions of the body portion moving across the body wall to generate a plurality of sub-portion tracking results; and
the processing unit is configured to generate the tracking result based on the plurality of sub-portion tracking results.

7. (canceled)

8. The system of claim 1, wherein the processing unit is further configured to:
determine a measurement of the object after the object has moved across the body wall; and
generate the tracking result based on the measurement.

9.-10. (canceled)

11. The system of claim 8, wherein the processing unit is further configured to:
in response to determining that the object includes a plurality of sub-portions of a body portion, determine the measurement based on an aggregation of individual measurements of sub-portions of the plurality of sub-portions; or
generate a measurement difference by comparing the measurement of the object with a reference measurement, the reference measurement being of the object before the object moves across the body wall, and generate the tracking result by generating a confirmation based on the measurement difference, the confirmation indicating whether an entirety of the object has moved across the body wall or whether an integrity of the object after the object has moved across the body wall.

12.-13. (canceled)

14. The system of claim 1, wherein the tracking result includes a path of the object moving across the body wall.

15. The system of claim 1, wherein the processing unit is further configured to:
determine a second registration between the first image sensor and a common reference;
determine a third registration between the second image sensor and the common reference; and
cause to be displayed on a display, a representation of the first image data and second image data transformed to the common reference based on the second registration and the third registration respectively.

16. (canceled)

17. The system of claim 1, further comprising a manipulator assembly, wherein the processing unit is further configured to: operate the tool using the manipulator assembly based on the tracking result.

18. (canceled)

19. The system of claim 1, wherein the tracking results are used to determine an operation status associated with performing an operation associated with the object.

20. An object tracking method, comprising:
receiving first image data from a first image sensor exterior to a body, the first image data including data of an object;
receiving second image data from a second image sensor interior to the body, the second image data including data of the object;
determining a first registration between the first image sensor and the second image sensor; and
generating a tracking result by tracking the object moving across a body wall of the body based on the first image data, the second image data, and the first registration, the tracking result indicating a status of movement of the object across the body wall.

21. (canceled)

22. The method of claim 20, further comprising:
determining a tool tracking configuration in response to determining that the object includes a tool for performing an operation in the body, the tool tracking configuration including a tool tracking step to track the tool moving in an entering-body direction or an exiting-body direction, the entering-body direction being from exterior to the body to interior to the body, and the exiting-body direction being from the interior to the body to the exterior to the body;
wherein tracking the object comprises performing at least the tool tracking step; and
wherein generating the tracking result is based on the tool tracking configuration.

23.-24. (canceled)

25. The method of claim 20, further comprising:
determining a measurement of the object after the object has moved across the body wall, wherein generating the tracking result comprises using the measurement.

26. (canceled)

27. The method of claim 20, further comprising:
generating a measurement difference by comparing the measurement of the object with a reference measurement, the reference measurement being of the object before the object moves across the body wall; wherein
generating the tracking result comprises using the measurement difference.

28. The method of claim 20, further comprising:
determining a second registration between the first image sensor and a common reference;
determining a third registration between the second image sensor and the common reference; and
causing to be displayed on a display, a representation of the first image data and second image data transformed to the common reference based on the second and third registrations respectively.

29. (canceled)

30. A non-transitory machine-readable medium comprising a plurality of machine-readable instructions which, when executed by one or more processors of a tracking system, are adapted to cause the one or more processors to perform a method comprising:
receiving first image data from a first image sensor exterior to a body, the first image data including data of an object;
receiving second image data from a second image sensor interior to the body, the second image data including data of the object;
determining a first registration between the first image sensor and the second image sensor; and
generating a tracking result by tracking the object moving across a body wall of the body based on the first image data, the second image data, and the first registration, the tracking result indicating a status of movement of the object across the body wall.

31. (canceled)

32. The non-transitory machine-readable medium of claim 30, further comprising:
determining a measurement of the object after the object has moved across the body wall, wherein generating the tracking result comprises using the measurement.

33. The non-transitory machine-readable medium of claim 32, further comprising:
generating a measurement difference by comparing the measurement of the object with a reference measurement, the reference measurement being of the object before the object moves across the body wall; wherein
generating the tracking result comprises using the measurement difference.

34. The non-transitory machine-readable medium of claim 30, wherein the method further comprises:
operating a tool using a manipulator assembly based on the tracking result.
